(19) United States
(12) Patent Application Publication
(10) Pub. No.: US 2024/0415746 A1
(43) Pub. Date: Dec. 19, 2024

(54) Oral Care Compositions

(71) Applicant: Colgate-Palmolive Company, New York, NY (US)

(72) Inventors: Joshua Eric KAUFFMAN, Hillsborough, NJ (US); Qichao RUAN, Hillsborough, NJ (US); Nihal DOGU, Dayton, NJ (US); Stacey LAVENDER, Chesterfield, NJ (US); Betty WON, Princeton Junction, NJ (US); Michael STRANICK, Bridgewater, NJ (US); Shiyou XU, Princeton, NJ (US); Luciana RINAUDI MARRON, Somerset, NJ (US); Dennis ZHANG, Piscataway, NJ (US)

(73) Assignee: Colgate-Palmolive Company, New York, NY (US)

(21) Appl. No.: 18/744,029

(22) Filed: Jun. 14, 2024

Related U.S. Application Data
(60) Provisional application No. 63/508,656, filed on Jun. 16, 2023.

(51) Int. Cl. A61K 8/21; A61K 8/24; A61K 8/81; A61K 8/97; A61Q 11/00

(52) U.S. Cl. CPC A61K 8/21; A61K 8/24; A61K 8/8147; A61K 8/8176; A61K 8/8182; A61K 8/97; A61Q 11/00

(57) ABSTRACT

Oral care compositions and, e.g., to oral care compositions adapted to form a film on an enamel substrate are disclosed herein. In accordance with an aspect, provided is a method having from about 1 to about 10 wt. % of a fluoride ion source; optionally, from about 2 to about 20 wt. % of hydroxyapatite; from about 3 to about 30 wt. % of a film-forming agent; and from about 7 to about 35 wt. % of an adhesive, wherein the oral care composition is adapted to form a film on teeth, and all weight percentages are based on the total weight of the oral care composition.

### CROSS-REFERENCE TO RELATED APPLICATION

This application claims the benefit of priority from U.S. Provisional Application No. 63/508,656, filed Jun. 16, 2023, the contents of which are hereby incorporated herein by reference in their entirety.

### BACKGROUND

Dental enamel is a thin, hard layer of calcified material that covers the crown of teeth. Dental enamel is the first line of defense for tooth protection against acid and physical challenges. The major mineral component of dental enamel is hydroxyapatite, a crystalline form of calcium phosphate. Dental enamel is formed by 7 hierarchical levels of hydroxyapatite microstructures. The hierarchical organization of hydroxyapatite crystals enable the robust mechanical properties of enamel. Mature enamel does not contain cells and thus cannot regenerate unlike other biomaterials such as bone and dentine.

Dental erosion occurs initially in the enamel and, if unchecked, may proceed to the underlying dentin. Dental erosion may be caused or exacerbated by acidic foods and drinks, and stomach acids arising from gastric reflux. Depending upon the relative pH of surrounding saliva, the tooth enamel will lose or gain positively charged ions such as calcium ions. Generally, saliva has a pH between 6.7 to 7.4. When the pH is lowered and concentration of hydrogen ions becomes relatively high, it damages the enamel and creates a porous, sponge-like roughened surface. The erosion of dental enamel can lead to enhanced tooth sensitivity due to increased exposure of the dentin tubules and increased dentin visibility leading to the appearance of more yellow teeth. In addition, when enamel erodes, the tooth is more susceptible to cavities or tooth decay.

Early acid damage on enamel is reversible by remineralization, in which mineral ions from saliva are reintroduced into the demineralized enamel. It has been reported that hydroxyapatite possesses a remineralizing effect on teeth and can be used to reduce tooth sensitivity.

Fluoride varnishes have been used in an attempt to reduce dental erosion. However, many conventional fluoride varnishes leave a long lasting hard coat on the teeth that must be broken and picked or brushed from the teeth. Moreover, many conventional fluoride varnishes may have a yellow color or other properties that make them not aesthetically pleasing to the patient.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions and, e.g., to oral care compositions adapted to form a film on an enamel substrate (e.g., teeth). In accordance with an aspect of the invention, provided is an oral care composition having from about 1 to about 10 wt. % of a fluoride source (e.g., a fluoride ion source); from about 2 to about 20 wt. % of hydroxyapatite; from about 3 to about 30 wt. % of a film-forming agent; and from about 7 to about 35 wt. % of an adhesive, wherein the oral care composition is adapted to form a film on teeth, and wherein all weight percentages are based on the total weight of the oral care composition.

A list of non-limiting example embodiments in accordance with various aspects of the invention is provided below.

In accordance with an embodiment 1, provided is an oral care composition including from about 1 to about 10 wt. % of a fluoride ion source; from about 2 to about 20 wt. % of hydroxyapatite; from about 3 to about 30 wt. % of a film-forming agent; and from about 7 to about 35 wt. % of an adhesive, wherein the oral care composition is adapted to form a film on teeth, and all weight percentages are based on the total weight of the oral care composition.

In accordance with an embodiment 2, provided is the oral care composition according to claim 1, wherein the fluoride ion source is selected from sodium fluoride, potassium fluoride, sodium monofluorophosphate, sodium fluorosilicate, ammonium fluorosilicate, amine fluoride, ammonium fluoride, and a combination of two or more thereof.

In accordance with an embodiment 3, provided is the oral care composition according to embodiment 1 or embodiment 2, wherein the oral care composition delivers from about 5,000 to about 48,000 ppm of fluoride ions.

In accordance with an embodiment 4, provided is the oral care composition according to according to any foregoing embodiment, wherein the film-forming agent is selected from an acrylamide polymer, an acrylate polymer, a cellulose, a cellulose derivative, a gum, a pyrrolidone polymer, and a combination of two or more thereof.

In accordance with an embodiment 5, provided is the oral care composition according to any foregoing embodiment, wherein the hydroxyapatite comprises nano-hydroxyapatite.

In accordance with an embodiment 6, provided is the oral care composition according to any of embodiment 1 to embodiment 5, wherein the hydroxyapatite comprises micro-hydroxyapatite.

In accordance with an embodiment 7, provided is the oral care composition according to any foregoing embodiment, wherein the hydroxyapatite is functionalized.

In accordance with an embodiment 8, provided is the oral care composition according to any foregoing embodiment, wherein the film-forming agent comprises a hydroxypropylmethyl cellulose, hydroxyethyl cellulose, hydroxypropyl cellulose, polyvinyl pyrrolidone, carboxymethyl cellulose, sodium carboxy methyl cellulose, methyl cellulose, polyvinyl alcohol, sodium alginate, polyethylene glycol, polyethylene oxide, chitosan, xanthan gum, tragacantha, guar gum, acacia gum, arabic gum, carrageenan, pululan, polyacrylic acid, methylmethacrylate copolymer, carboxyvinyl copolymers, octylacrylamide copolymer, or a combination of two or more thereof.

In accordance with an embodiment 9, provided is the oral care composition according to any foregoing embodiment, wherein the film-forming agent comprises a polyacrylate copolymer.

In accordance with an embodiment 10, provided is the oral care composition according to any foregoing embodiment, wherein the adhesive is selected from a rosin, a polyvinyl acetaldehyde, a polyvinyl alcohol, a polyvinyl acetate, a poly(ethylene oxide), a polyacrylate, a polyvinylpyrrolidone, a polyvinylpyrrolidone/vinyl acetate copolymer, a polyoxyethylene/polyoxypropylene block copolymer, a silicone resin, an organo phosphoric acid having alkyl group, and a combination of two or more thereof.

In accordance with an embodiment 11, provided is the oral care composition according to embodiment 10, wherein the rosin is selected from a wood rosin, a gum rosin, a tall oil rosin, and a combination of two or more thereof.

In accordance with an embodiment 12, provided is the oral care composition according to embodiment 10 or embodiment 11, wherein the rosin is at least partially hydrogenated or fully hydrogenated.

In accordance with an embodiment 13, provided is the oral care composition according to one of embodiment 10 to embodiment 12, wherein the adhesive comprises a polyvinylpyrrolidone, a polyvinylpyrrolidone/vinyl acetate copolymer, or a combination thereof.

In accordance with an embodiment 14, provided is the oral care composition according to any foregoing embodiment further comprising: from about 20 to about 70 wt. % of an orally acceptable vehicle.

In accordance with an embodiment 15, provided is the oral care composition according to embodiment 14, wherein the orally acceptable vehicle comprises a volatile component.

In accordance with an embodiment 16, provided is the oral care composition according to embodiment 15, wherein the volatile component is selected from one or more of ethyl acetate i-pentyl propionate, acetone, ethylacetate i-amylpropionate, hydrogen peroxide, methanol, ethanol, propanol, isopropanol, butanol, and a combination of two or more thereof.

In accordance with an embodiment 17, provided is the oral care composition according to embodiment 15 or embodiment 16, wherein the volatile component is present in an amount from about 4 to about 65 wt. %.

In accordance with an embodiment 18, provided is the oral care composition according to any foregoing embodiment further comprising: one or more plasticizer.

In accordance with an embodiment 19, provided is the oral care composition according to embodiment 18, wherein the one or more plasticizer is selected from citric acid esters, polyethylene glycol (PEG) polypropylene glycol (PPG), PEG/PPG, phthalic esters, phosphoric esters, esters of adipic, azelaic, glutamic acid, sebacic acid, fatty acid esters citric esters, esters of acetic, propionic or butyric acid, esters of ethylbutyric or ethyl hexanoic acid, glycol esters, benzoic esters, trimellitic esters, sulfonic esters, sulfonamides, anilides, alcohols, ethers, ketones, and a combination of two or more thereof.

In accordance with an embodiment 20, provided is the oral care composition according to embodiment 18 or embodiment 19, wherein the plasticizer comprises a citric acid ester.

In accordance with an embodiment 21, provided is the oral care composition according to embodiment 20, wherein the citric acid ester is selected from acetyl tributyl citrate, acetyl triethyl citrate, and a combination of two or more thereof.

In accordance with an embodiment 22, provided is the oral care composition according to any foregoing embodiment, wherein the oral care composition comprises a weight ratio of the film-forming agent to the adhesive from about 1:1 to about 1:5.

In accordance with an embodiment 23, provided is the oral care composition according to any foregoing embodiment, wherein the oral care composition comprises about 4 wt. % or less of a sweetener.

In accordance with an embodiment 24, provided is the oral care composition according to any foregoing embodiment, wherein the oral care composition comprises about 4 wt. % or less of a sugar alcohol.

In accordance with an embodiment 25, provided is the oral care composition according to any foregoing embodiment, wherein the oral care composition comprises about 4 wt. % or less of a surfactant.

In accordance with an embodiment 26, provided is the oral care composition according to any foregoing embodiment, wherein the oral care composition comprises about 4 wt. % or less of water.

In accordance with an embodiment 27, provided is the oral care composition according to any foregoing embodiment, wherein the oral care composition is substantially free or free of water.

In accordance with an embodiment 28, provided is the oral care composition according to any foregoing embodiment, wherein the oral care composition is adapted to form a film on an enamel substrate within 2 minutes from application to the enamel substrate.

In according with an embodiment 29, provided is a method for forming a film on an enamel substrate, the method including: rinsing an enamel substrate; drying the enamel substrate; and applying the oral care composition according to any foregoing embodiment to the enamel substrate.

In according with an embodiment 30, provided is an oral care composition including: from about 1 to about 10 wt. % of a fluoride ion source; from about 3 to about 30 wt. % of a film-forming agent; and from about 7 to about 35 wt. % of an adhesive, wherein the oral care composition is adapted to form a film on teeth, and all weight percentages are based on the total weight of the oral care composition.

In accordance with an embodiment 31, provided is the oral care composition according to embodiment 30, wherein the fluoride ion source is selected from sodium fluoride, potassium fluoride, sodium monofluorophosphate, sodium fluorosilicate, ammonium fluorosilicate, amine fluoride, ammonium fluoride, and a combination of two or more thereof.

In accordance with an embodiment 32, provided is the oral care composition according to embodiment 30 or embodiment 31, wherein the oral care composition delivers from about 5,000 to about 48,000 ppm of fluoride ions.

In accordance with an embodiment 33, provided is the oral care composition according to one of embodiment 30 to embodiment 32, wherein the film-forming agent comprises a hydroxypropylmethyl cellulose, hydroxyethyl cellulose, hydroxypropyl cellulose, polyvinyl pyrrolidone, carboxymethyl cellulose, sodium carboxy methyl cellulose, methyl cellulose, polyvinyl alcohol, sodium alginate, polyethylene glycol, polyethylene oxide, chitosan, xanthan gum, tragacantha, guar gum, acacia gum, arabic gum, carrageenan, pululan, polyacrylic acid, methylmethacrylate copolymer, carboxyvinyl copolymers, octylacrylamide copolymer, or a combination of two or more thereof.

In accordance with an embodiment 34, provided is the oral care composition according to one of embodiment 30 to embodiment 33, wherein the film-forming agent comprises a polyacrylate copolymer.

In accordance with an embodiment 35, provided is the oral care composition according to one of embodiment 30 to embodiment 34, wherein the adhesive is selected from a rosin, a polyvinyl acetaldehyde, a polyvinyl alcohol, a polyvinyl acetate, a poly(ethylene oxide), a polyacrylate, a polyvinylpyrrolidone, a polyvinylpyrrolidone/vinyl acetate copolymer, a polyoxyethylene/polyoxypropylene block copolymer, a silicone resin, an organo phosphoric acid having alkyl group, and a combination of two or more thereof.

In accordance with an embodiment 36, provided is the oral care composition according to embodiment 35, wherein the adhesive comprises a polyvinylpyrrolidone.

In accordance with an embodiment 37, provided is the oral care composition according to embodiment 34 or embodiment 35, wherein the adhesive comprises a rosin selected from a wood rosin, a gum rosin, a tall oil rosin, and a combination of two or more thereof.

In accordance with an embodiment 38, provided is the oral care composition according to embodiment 37, wherein the rosin is at least partially hydrogenated or fully hydrogenated.

In accordance with an embodiment 39, provided is the oral care composition according to one of embodiment 30 to embodiment 38 further comprising: from about 20 to about 70 wt. % of an orally acceptable vehicle.

In accordance with an embodiment 40, provided is the oral care composition according to embodiment 39, wherein the orally acceptable vehicle comprises a volatile component.

In accordance with an embodiment 41, provided is the oral care composition according to embodiment 40, wherein the volatile component comprises ethyl acetate i-pentyl propionate, acetone, ethylacetate i-amylpropionate, hydrogen peroxide, methanol, ethanol, propanol, isopropanol, butanol, and a combination of two or more thereof.

In accordance with an embodiment 42, provided is the oral care composition according to embodiment 40 or embodiment 41, wherein the volatile component is present in an amount from about 4 to about 65 wt. %.

In accordance with an embodiment 43, provided is the oral care composition according to one of embodiment 30 to embodiment 42 further comprising: one or more plasticizer.

In accordance with an embodiment 44, provided is the oral care composition according to embodiment 43, wherein the one or more plasticizer is selected from citric acid esters, polyethylene glycol (PEG) polypropylene glycol (PPG), PEG/PPG, phthalic esters, phosphoric esters, esters of adipic, azelaic, glutamic acid, sebacic acid, fatty acid esters citric esters, esters of acetic, propionic or butyric acid, esters of ethylbutyric or ethyl hexanoic acid, glycol esters, benzoic esters, trimellitic esters, sulfonic esters, sulfonamides, anilides, alcohols, ethers, ketones, and a combination of two or more thereof.

In accordance with an embodiment 45, provided is the oral care composition according to embodiment 43 or embodiment 44, wherein the plasticizer comprises a citric acid ester.

In accordance with an embodiment 46, provided is the oral care composition according to embodiment 44, wherein the citric acid ester is selected from acetyl tributyl citrate, acetyl triethyl citrate, and a combination of two or more thereof.

In accordance with an embodiment 47, provided is the oral care composition according to one of embodiment 30 to embodiment 46, wherein the oral care composition comprises a weight ratio of the film-forming agent to the adhesive from about 1:1 to about 1:5.

In accordance with an embodiment 48, provided is the oral care composition according to one of embodiment 30 to embodiment 47, wherein the oral care composition comprises about 4 wt. % or less of a sweetener.

In accordance with an embodiment 49, provided is the oral care composition according to one of embodiment 30 to embodiment 48, wherein the oral care composition comprises about 4 wt. % or less of a sugar alcohol.

In accordance with an embodiment 50, provided is the oral care composition according to one of embodiment 30 to embodiment 49, wherein the oral care composition comprises about 4 wt. % or less of a surfactant.

In accordance with an embodiment 51, provided is the oral care composition according to one of embodiment 30 to embodiment 50, wherein the oral care composition comprises about 4 wt. % or less of water.

In accordance with an embodiment 52, provided is the oral care composition according to one of embodiment 30 to embodiment 51, wherein the oral care composition is substantially free or free of water.

In accordance with an embodiment 53, provided is the oral care composition according to one of embodiment 30 to embodiment 52, wherein the oral care composition is adapted to form a film on an enamel substrate within 2 minutes from application to the enamel substrate.

In accordance with an embodiment 54, provided is a method for forming a film on an enamel substrate, the method comprising: rinsing an enamel substrate; drying the enamel substrate; and applying the oral care composition according to one of embodiment 29 to embodiment 51 to the enamel substrate.

In accordance with an embodiment 55, an oral care composition comprising: from about 1 to about 10 wt. % of a fluoride ion source; optionally, from about 2 to about 20 wt. % of hydroxyapatite; from about 3 to about 30 wt. % of a film-forming agent; from about 7 to about 35 wt. % of an adhesive; and a polyvinylpyrrolidone, wherein the oral care composition is adapted to form a film on teeth, and all weight percentages are based on the total weight of the oral care composition.

In accordance with an embodiment 56, provided is the oral care composition according to embodiment 55, wherein the polyvinylpyrrolidone is present in an amount from about 0.5 to about 5 wt. %.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is scanning electron microscopy images of enamel blocks receiving a non-limiting example of an oral care composition or a comparative composition in accordance with aspects of the invention;

FIG. 2 is cross-sectional views of scanning electron microscopy images for a sound enamel block, an acid damaged enamel block, and an enamel block receiving a non-limiting example of an oral care composition in accordance with aspects of the invention;

FIG. 3 is a bar graph of the soluble fluoride uptake exhibited by a non-limiting example of an oral care composition or a comparative composition in accordance with aspects of the invention;

FIG. 4 is a bar graph of the structurally bound fluoride uptake exhibited by a non-limiting example of an oral care composition or a comparative composition in accordance with aspects of the invention;

FIG. 5 is top and cross-sectional views of scanning electron microscopy images for enamel blocks receiving a non-limiting example of an oral care composition or a comparative composition in accordance with aspects of the invention; and

FIG. 6 is a bar graph of the fluoride uptake by oral care compositions in accordance with aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges, such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition, unless indicated otherwise. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═”, and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a fluoride ion source and a stannous ion source. If a particular oral care composition includes both a fluoride ion source and a stannous ion source, stannous fluoride will serve only as either a fluoride ion source or a stannous ion source—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions and, e.g., to oral care compositions adapted to form a film on an enamel substrate (e.g., teeth). In accordance with an aspect of the invention, provided is a method having from about 1 to about 10 wt. % of a fluoride ion source; from about 2 to about 20 wt. % of hydroxyapatite; from about 3 to about 30 wt. % of a film-forming agent; and from about 7 to about 35 wt. % of an adhesive, wherein the oral care composition is adapted to form a film on teeth, and all weight percentages are based on the total weight of the oral care composition.

The oral care compositions are typically adapted to form a film on an enamel substrate, such as a tooth. In some embodiments, the oral care composition is adapted to form a film on an enamel substrate within about 5 minutes of application to the enamel substrate. For example, the oral care composition may form a film in about 4 minute or less, about 3 minute or less, about 2 minute or less, about 1 minute or less, about 45 seconds or less, about 30 seconds or less, about 20 seconds or less, or about 10 seconds or less from application to the enamel substrate. In some embodiments, the oral care composition may be adapted to form a film in about 10 seconds to about 3 minutes, about 10 seconds to about 2 minutes, about 10 seconds to about 1 minute; from about 20 seconds to about 3 minutes, about 20 seconds to about 2 minutes, about 20 seconds to about 1 minute; from about 30 seconds to about 3 minutes, about 30 seconds to about 2 minutes, or about 30 seconds to about 1 minute. In other embodiments, the oral care composition may be adapted to form a film in about 1 to about 4 minutes, about 1 to about 3 minutes, about 1 to about 2 minutes; from about 2 to about 4 minutes, about 2 to about 3 minutes, or any range or subrange thereof.

The oral care compositions may, in some embodiments, be preferably adapted to have a reduced amount of sugar alcohols, sweetener, surfactant, and/or water. For example, the oral care composition may have about 4 wt. % or less of sugar alcohol(s), based on the total weight of the oral care composition. In certain embodiments, the oral care composition has a total amount of sugar alcohol(s) of about 3 wt. % or less, about 2 wt. % or less, about 1 wt. % or less, about 0.5 wt. % or less, or about 0.1 wt. % or less, based on the total weight of the oral care composition.

Additionally or alternatively, the oral care composition may have about 4 wt. % or less of sweetener(s), based on the total weight of the oral care composition. For example, the oral care composition may have a total amount of sweetener(s) of about 3 wt. % or less, about 2 wt. % or less, about 1 wt. % or less, about 0.5 wt. % or less, or about 0.1 wt. % or less, based on the total weight of the oral care composition.

The oral care composition may also have in some instances about 4 wt. % or less of surfactant(s), based on the total weight of the oral care composition. For instance, the oral care composition may have a total amount of surfactant of about 3 wt. % or less, about 2 wt. % or less, about 1 wt. % or less, about 0.5 wt. % or less, or about 0.1 wt. % or less, based on the total weight of the oral care composition.

In some embodiments, the oral care composition may have about 4 wt. % or less of water, based on the total weight of the oral care composition. For example, the oral care composition may have a total amount of water of about 3 wt. % or less, about 2 wt. % or less, about 1 wt. % or less, about 0.5 wt. % or less, or about 0.1 wt. % or less, based on the total weight of the oral care composition.

Suitable components, such as those listed below, may be included or excluded from the formulations for the oral care compositions depending on the specific combination of other ingredients and the form of the oral care compositions. Additionally or alternatively, the oral care compositions may in some embodiments have a single phase, which contains components and/or ingredients of the oral care composition. In other embodiments, the oral care composition may include two or more phases.

The oral care composition typically comprises a fluoride source (e.g., a fluoride ion source), preferably in an amount from about 1 to about 10 wt. %, based on the total weight of the oral care composition. In some embodiments, the oral care composition includes a fluoride source (e.g., a fluoride ion source) in an amount from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 5 to about 10 wt. %, about 5 to about 8 wt. %, about 5 to about 7 wt. %; from about 7 to about 10 wt. %, about 7 to about 9 wt. %, or any range or subrange thereof, based on the total weight of the oral care composition.

The fluoride source and/or fluoride ion source may be selected from soluble fluoride ion salts. For example, the fluoride source and/or fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminocthyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride source and/or fluoride ion source comprises sodium fluoride, sodium monofluorophosphate, or a combination thereof. Additional examples of fluoride ion sources are disclosed in U.S. Pat. Nos. 3,535,421, 4,885,155, and 3,678,154, the disclosure of each of which is hereby incorporated by reference in their entirety.

In certain embodiments, the oral care composition of the disclosure may contain a source of fluoride ions or fluorine-providing agents (e.g., fluoride ion source in an amount sufficient to deliver (e.g., to an enamel substrate, such as teeth), in total, from about 5,000 ppm to about 48,000 ppm (mass fraction) of fluoride ions, generally at least 10,000 ppm, e.g., from about 10,000 to about 40,000 ppm, about 15,000 to about 35,000 ppm, about 18,000 to about 30,000 ppm, about 19,000 to about 27,000 ppm, about 20,000 to about 25,000 ppm, about 21,000 to about 24,000 ppm, or any range or subrange thereof.

The oral care compositions may include hydroxyapatite. The amount of hydroxyapatite in the oral care composition may be from about 2 to about 20 wt. %, based on the total weight of the oral care composition. For instance, the hydroxyapatite may be present in the oral care composition in an amount from about 2 to about 17 wt. %, about 2 to about 15 wt. %, about 2 to about 13 wt. %, about 2 to about 11 wt. %, about 2 to about 9 wt. %, about 2 to about 7 wt. %, about 2 to about 6 wt. %; from about 4 to about 20 wt. %, about 4 to about 17 wt. %, about 4 to about 15 wt. %, about 4 to about 13 wt. %, about 4 to about 11 wt. %, about 4 to about 9 wt. %, about 4 to about 7 wt. %, about 4 to about 6 wt. %; from about 6 to about 20 wt. %, about 6 to about 17 wt. %, about 6 to about 15 wt. %, about 6 to about 13 wt. %, about 6 to about 11 wt. %, about 6 to about 9 wt. %; from about 8 to about 20 wt. %, about 8 to about 17 wt. %, about 8 to about 15 wt. %, about 8 to about 13 wt. %, about 8 to about 11 wt. %, about 8 to about 10 wt. %; from about 10 to about 20 wt. %, about 10 to about 17 wt. %, about 10 to about 15 wt. %, about 10 to about 13 wt. %; from about 12 to about 20 wt. %, about 12 to about 17 wt. %, about 12 to about 15 wt. %; from about 15 to about 20 wt. %, about 15 to about 17 wt. %, about 17 to about 20 wt. %, or any range or subrange thereof, based on the total weight of the oral care composition.

The hydroxyapatite may be selected from micro-hydroxyapatite, nano-hydroxyapatite, or a combination thereof. The micro-hydroxyapatite may have a mean diameter of greater than 1 μm and up to 600 μm. For example, the micro-hydroxyapatite may have an average diameter of about 1 to about 600 μm, about 1 to about 500 μm, about 1 to about 400 μm, about 1 to about 300 μm, about 1 to about 200 μm, about 1 to about 100 μm; from about 5 to about 600 μm, about 5 to about 500 μm, about 5 to about 400 μm, about 5 to about 300 μm, about 5 to about 200 μm, about 5 to about 100 μm; from about 30 to about 600 μm, about 30 to about 500 μm, about 30 to about 400 μm, about 30 to about 300 μm, about 30 to about 200 μm, about 30 to about 100 μm; from about 60 to about 600 μm, about 60 to about 500 μm, about 60 to about 400 μm, about 60 to about 300 μm, about 60 to about 200 μm, about 60 to about 100 μm; from about 90 to about 600 μm, about 90 to about 500 μm, about 90 to about 400 μm, about 90 to about 300 μm, about 90 to about 200 μm, about 90 to about 100 μm; from about 150 to about 600 μm, about 150 to about 500 μm, about 150 to about 400 μm, about 150 to about 300 μm; from about 250 to about 600 μm, about 250 to about 500 μm, about 200 to about 400 μm, or any range or subrange thereof.

The hydroxyapatite may be selected from nano-hydroxyapatite having a mean diameter of less than 1000 nm, e.g., about 1 to about 950 nm, about 50 to about 950 nm, about 100 nm to about 950 nm, about 200 nm to 950 nm, about 400 nm to 950 nm, about 600 nm to 950 nm, about 800 nm to 950 nm; from about 1 to about 700 nm, about 50 to about 700 nm, about 100 nm to about 700 nm, about 200 nm to 700 nm, about 400 nm to 700 nm, about 600 nm to 700 nm; about 1 to about 500 nm, about 50 to about 500 nm, about 100 nm to about 500 nm, about 200 nm to 500 nm, about 400 nm to 500 nm; about 1 to about 300 nm, about 50 to about 300 nm, about 100 nm to about 300 nm, about 200 nm to 300 nm; about 1 to about 100 nm, about 40 to about 100 nm, about 70 nm to about 100 nm, or any range or subrange thereof.

The hydroxyapatite may be a functionalized hydroxyapatite, e.g., HAP CaCO₃, ZnCO₃-hydroxyapatite, or HAP/TCP (tricalcium phosphate). In at least one embodiment, the functionalized hydroxyapatite is HAP/CaCO₃, whereby the HAP/CaCO₃ has a calcium carbonate core and hydroxyapatite outer shell. The hydroxyapatite outer shell may cover about 40% or more of the outer surface the core, e.g., from about 40 to about 95%, about 60 to about 95%, about 70 to about 95%, about 80 to about 95%, about 70 to 100%, about 80 to about 100%, or about 90 to about 100% of the outer surface of the core. For example, the oral care composition may comprise functionalized nano-hydroxyapatite, functionalized micro-hydroxyapatite, and/or functionalized hydroxyapatite having an average particle size of 1 nm or more.

The oral care compositions generally include one or more film forming agent(s). The film forming agent(s) may be present in an amount from about 3 to about 30 wt. %, based on the total weight of the oral care composition, based on the total weight of the oral care composition. In some embodiments, the total amount of film forming agent(s) in the oral care composition is from about 3 to about 30 wt. %, about 5 to about 30 wt. %, about 7 to about 30 wt. %, about 9 to about 30 wt. %, about 11 to about 30 wt. %, about 14 to about 30 wt. %, about 17 to about 30 wt. %, about 20 to about 30 wt. %, about 24 to about 30 wt. %; from about 3 to about 25 wt. %, about 5 to about 25 wt. %, about 7 to about 25 wt. %, about 9 to about 25 wt. %, about 11 to about 25 wt. %, about 14 to about 25 wt. %, about 17 to about 25 wt. %, about 20 to about 25 wt. %; from about 3 to about 20 wt. %, about 5 to about 20 wt. %, about 7 to about 20 wt. %, about 9 to about 20 wt. %, about 11 to about 20 wt. %, about 14 to about 20 wt. %, about 17 to about 20 wt. %; from about 3 to about 16 wt. %, about 5 to about 16 wt. %, about 7 to about 16 wt. %, about 9 to about 16 wt. %, about 11 to about 16 wt. %; from about 3 to about 12 wt. %, about 5 to about 12 wt. %, about 7 to about 12 wt. %, about 9 to about 12 wt. %; from about 3 to about 9 wt. %, about 5 to about 9 wt. %, about 7 to about 9 wt. %; from about 3 to about 7 wt. %, about 5 to about 7 wt. %, about 3 to about 5 wt. %, or any range or subrange thereof, based on the total weight of the oral care composition.

Film forming agents generally refer to polymers, prepolymers, and/or monomers capable of forming alone, or in the presence of at least one additional agent, a continuous and adherent film on a tooth substrate. The term “prepolymer” refers to a system of monomers that have been reacted to an intermediate molecular mass state. This material is capable of further polymerization by reactive groups to reach a fully cured high molecular weight state. As such, mixtures of reactive polymers with unreacted monomers may also be referred to as prepolymers.

The film forming agents may be selected from water-soluble, water-dispersible, water-swellable, water-insoluble, or combinations thereof, and may include cellulose or cellulose derivatives. Although the use of water-swellable and water-insoluble polymers is contemplated, the formulations may, also or alternatively, include a sufficient amount of water-soluble polymer and/or water-dispersible polymer to ensure the eventual disintegration of the film formed from the oral care composition. As examples of various potentially suitable film forming agents, cellulose derivatives, polyvinyl compounds, styrol-butadiene-acrylate copolymers, polyacrylates, and polyamides are worth mentioning.

Examples of film forming agents include polymers, such as water-soluble hydroxypropylmethyl cellulose, hydroxyethyl cellulose, hydroxypropyl cellulose, polyvinyl pyrrolidone, carboxymethyl cellulose, sodium carboxymethyl cellulose, methyl cellulose, polyvinyl alcohol, sodium alginate, polyethylene glycol, polyethylene oxide, chitosan, xanthan gum, tragacantha, guar gum, acacia gum, arabic gum, carrageenan, pululan, polyacrylic acid, methylmethacrylate copolymer, carboxyvinyl copolymers, various copolymer or combinations thereof, cellulose derivatives, gums, or combinations of two or more thereof. Other polymers that may be used include, but are not limited to, ethyl cellulose, hydroxypropyl ethyl cellulose, cellulose acetate phthalate, hydroxypropyl methyl cellulose phthalate, copolymers thereof, and combinations of two or more thereof.

In certain embodiments, the film forming agent is selected from methacrylate copolymers. The methacrylate copolymers may be adapted to have chemical and physical properties, such as: a molecular weight of about 47,000 g/mol, a refractive index of about 1.380 to about 1.385, an alkali value of about 180 mg KOH/g of copolymer, and a glass transition temperature of about 45° C.

In further embodiments, the film forming agent is selected from acrylate/octylacrylamide copolymer. Acrylate/octylacrylamide copolymer typically include a copolymer of octylacrylamide (e.g., N-(1,1,3,3-tetramethylbutyl)-2-propenamide) and one or more monomers selected from acrylic acid, methacrylic acid and their simple esters. In at least one embodiment, the acrylate/octylacrylamide copolymer is 2-propenoic acid, 2-methyl-, 2-methylpropyl ester, polymer with 2-propenoic acid and N-(1,1,3,3-tetramethylbutyl)-2-propenamide. The acrylate/octylacrylamide copolymer may be selected from 2-propenoic acid, 2-methyl-, 2-methylpropyl ester, polymer with 2-propenoic acid and N-(1,1,3,3-tetramethylbutyl)-2-propenamide, and a combination of two or more thereof.

One or more adhesives are typically included in the oral care composition. The one or more adhesives are generally present in an amount from about 7 to about 35 wt. %, based on the total weight of the oral care composition. For instance, the oral care composition may include adhesive(s) in a total amount from about 7 to about 32 wt. %, about 7 to about 29 wt. %, about 7 to about 26 wt. %, about 7 to about 23 wt. %, about 7 to about 20 wt. %, about 7 to about 17 wt. %, about 7 to about 14 wt. %, about 7 to about 11 wt. %; from about 10 to about 35 wt. %, about 10 to about 32 wt. %, about 10 to about 29 wt. %, about 10 to about 26 wt. %, about 10 to about 23 wt. %, about 10 to about 20 wt. %, about 10 to about 17 wt. %, about 10 to about 14 wt. %; from about 13 to about 35 wt. %, about 13 to about 32 wt. %, about 13 to about 29 wt. %, about 13 to about 26 wt. %, about 13 to about 23 wt. %, about 13 to about 20 wt. %; from about 16 to about 35 wt. %, about 16 to about 32 wt. %, about 16 to about 29 wt. %, about 16 to about 26 wt. %, about 16 to about 23 wt. %; from about 19 to about 35 wt. %, about 19 to about 32 wt. %, about 19 to about 29 wt. %, about 19 to about 26 wt. %, about 19 to about 23 wt. %; from about 22 to about 35 wt. %, about 22 to about 32 wt. %, about 22 to about 29 wt. %, about 22 to about 26 wt. %; from about 26 to about 35 wt. %, about 26 to about 32 wt. %, about 30 to about 35 wt. %, or any range or subrange thereof, based on the total weight of the oral care composition.

The one or more adhesive(s) may be selected from alkyd resins, polyvinyl acetaldehydes, polyvinyl alcohols, polyvinyl acetates, poly(ethylene oxide), polyacrylates, ketone resins, polyvinylpyrrolidone, polyvinylpyrrolidone/vinyl acetate copolymer, polyethylene glycols of 200 to 1000 molecular weight and polyoxyethylene/polyoxypropylene block copolymers (Polyox), and a combination of two or more thereof. The adhesives may comprise of organo phosphoric acid compounds having alkyl groups. The alkyl group of the organo phosphoric acid may include at least two carbon atoms, such as from 3 to about 30 carbon atoms.

In some embodiments, the adhesive comprises a silicone pressure sensitive adhesive (PSA). PSAs can be produced by condensing a silicone resin and an organosiloxane, such as a polydiorganosiloxane. In some embodiments, the silicone polymers are prepared by mixing a silanol terminated polydiorganosiloxane, such as polydimethyl siloxane, with a silanol-containing silicone resin, whereby the silanol groups of the polydiorganosiloxane undergo a condensation reaction with the silanol groups of the silicone resin so that the polydiorganosiloxane is lightly crosslinked by the silicone resin. A catalyst, for example, an alkaline material, such as ammonia, ammonium hydroxide or ammonium carbonate, can be mixed with the silanol-terminated polydiorganosiloxane and the silicone resin to promote crosslinking. By copolymerizing the silicone resin with the silanol terminated polydiorganosiloxane, a polymer with self-adhering properties and a soft elastomer matrix can be produced.

In some preferred embodiments, the one or more adhesive comprises a rosin. The rosin may in some instances be a synthetic resin or a natural resin. Suitable natural resins include, but are not limited to, shellac, rosins and combinations thereof. Non-limiting examples of rosins include wood rosin, gum rosin, tall oil rosin, and mixtures of two or more thereof. The rosins may be in a crude state or a refined state. Additionally or alternatively, the rosin may be at least partially hydrogenated.

Hydrogenated rosins are rosin acids or resin acids, which have had some of their carbon-carbon double bonds hydrogenated. This hydrogenation gives the rosins resistance to air oxidation. Rosin acids that can be hydrogenated can include, but are not limited to: abietic-type acids, such as abictic acid, dihydroabietic acid, neoabietic acid, palustric acid, levopimaric acid, and pimaric acid, such as pimaric acid and isopimaric acid.

The rosin may, in certain embodiments, include a substantial fraction of resin acid components that are isomeric with abictic acid. The rosin may comprise of dihydroabietic acid (C₂₀H₃₂O₂) and/or dehydroabietic acid (C₂₀H₂₈O₂). The rosin may have a density of about 1.07 to about 1.09 g/cm3 in some instances.

In some embodiments, the oral care composition may be formulated to have a weight ratio of a total amount of film-forming agent(s) to total amount of adhesive(s) from about 1:1 to about 1:5. The weight ratio of the total amount of film-forming agent(s) to the total amount of adhesive(s) may be from about 1:1 to about 1:4, about 1:1 to about 1:3, about 1:1 to about 1:2; from about 1:1 to about 1:5, about 1:2 to about 1:4, about 1:2 to about 1:3, or any range or subrange thereof.

The oral care compositions may, in some embodiments, comprise a polyvinylpyrrolidone in an amount ranging from about 0.5 to about 5 wt. %. For example, the oral care composition may comprise polyvinylpyrrolidone in an amount from about 0.5 to about 4 wt. %, about 0.5 to about 3 wt. %, about 0.5 to about 2.5 wt. %, about 0.5 to about 2 wt. %; from about 1 to about 5 wt. %, about 1 to about 4 wt. %, about 1 to about 3 wt. %, about 1 to about 2.5 wt. %, about 1 to about 2 wt. %; from about 1.5 to about 5 wt. %, about 1.5 to about 4 wt. %, about 1.5 to about 3 wt. %, about 1.5 to about 2.5 wt. %, about 1.5 to about 2 wt. %; from about 2 to about 5 wt. %, about 2 to about 4 wt. %, about 2 to about 3 wt. %, about 2 to about 2.5 wt. %; from about 2.5 to about 5 wt. %, about 2.5 to about 4 wt. %, about 2.5 to about 3 wt. %, or any range or subrange thereof, based on the total weight of the oral care composition.

The oral care composition may include an orally acceptable vehicle. The orally acceptable vehicle may be present in the oral care composition in an amount from about 20 to about 70 wt. %, based on the total weight of the oral care composition. In some instances, the oral care composition comprises a total amount of an orally acceptable vehicle ranging from about 30 to about 70 wt. %, about 40 to about 70 wt. %, about 45 to about 70 wt. %, about 50 to about 70 wt. %, about 55 to about 70 wt. %; from about 20 to about 65 wt. %, about 30 to about 65 wt. %, about 40 to about 65 wt. %, about 45 to about 65 wt. %, about 50 to about 65 wt. %, about 55 to about 65 wt. %; from about 20 to about 60 wt. %, about 30 to about 60 wt. %, about 40 to about 60 wt. %, about 45 to about 60 wt. %, about 50 to about 60 wt. %; from about 20 to about 55 wt. %, about 30 to about 55 wt. %, about 40 to about 55 wt. %, about 45 to about 55 wt. %, about 50 to about 55 wt. %; from about 20 to about 50 wt. %, about 30 to about 50 wt. %, about 40 to about 50 wt. %, about 45 to about 50 wt. %; from about 20 to about 45 wt. %, about 30 to about 45 wt. %, about 40 to about 45 wt. %; from about 20 to about 40 wt. %, about 30 to about 40 wt. %, about 20 to about 30 wt. %, or any range or subrange thereof, based on the total weight of the oral care composition.

As used herein, “orally acceptable” means that the composition and the components thereof are safe for use in the mouth at the levels required. The orally acceptable vehicle may include a volatile content and/or a non-volatile component. The non-volatile component may include one or more polyols. Examples of polyols include glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof. The polyol may be a humectant including, e.g., glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer, and a combination of two or more thereof.

The volatile component may include ethanol, methanol, isopropanol, butanol, water, acetone, methyl ethyl ketone, ethyl acetate, butyl acetate, cyclohexanone, cyclohexane, methylene chloride, chloroform, carbon tetrachloride, trichloroethylene, perchloroethylene, methyl glycol acetate, i-pentyl propionate, ethyl acetate i-amylpropionate, hydrogen peroxide, toluene, benzene, diethyl ether, benzyl alcohol, flavor oil, or combinations thereof. For example, the volatile component may be selected from ethyl acetate i-pentyl propionate, acetone, ethyl acetate i-amylpropionate, hydrogen peroxide, methanol, ethanol, propanol, isopropanol, butanol, and a combination of two or more. In some embodiments, the volatile component comprises ethanol.

The volatile component may be present in an amount from about 4 to about 65 wt. %, based on the total weight of the oral care composition. For example, the oral care composition may include the volatile component in an amount from about 10 to about 65 wt. %, about 20 to about 65 wt. %, about 30 to about 65 wt. %, about 40 to about 65 wt. %, about 45 to about 65 wt. %, about 50 to about 65 wt. %, about 55 to about 65 wt. %; from about 10 to about 60 wt. %, about 20 to about 60 wt. %, about 30 to about 60 wt. %, about 40 to about 60 wt. %, about 45 to about 60 wt. %, about 50 to about 60 wt. %, about 55 to about 60 wt. %; from about 10 to about 55 wt. %, about 20 to about 55 wt. %, about 30 to about 55 wt. %, about 40 to about 55 wt. %, about 45 to about 55 wt. %, about 50 to about 55 wt. %; from about 10 to about 50 wt. %, about 20 to about 50 wt. %, about 30 to about 50 wt. %, about 40 to about 50 wt. %, about 45 to about 50 wt. %; from about 10 to about 45 wt. %, about 20 to about 45 wt. %, about 30 to about 45 wt. %, about 40 to about 45 wt. %; from about 10 to about 35 wt. %, about 20 to about 35 wt. %, about 30 to about 35 wt. %; from about 4 to about 25 wt. %, about 10 to about 25 wt. %, about 15 to about 25 wt. %, or any range or subrange thereof, based on the total weight of the oral care composition. The volatile component may comprise a portion or all of the orally acceptable vehicle.

The oral care composition may include one or more plasticizer(s), e.g., in an amount from about 1 to about 10 wt. %, based on the total weight of the oral care composition. For example, the oral care composition have a total amount of plasticizer(s) from about 1 to about 10 wt. %, about 1 to about 8 wt. %, about 1 to about 6 wt. %, about 1 to about 4 wt. %, about 1 to about 3 wt. %; from about 3 to about 10 wt. %, about 3 to about 8 wt. %, about 3 to about 6 wt. %, about 3 to about 5 wt. %; from about 5 to about 10 wt. %, about 5 to about 8 wt. %, about 5 to about 7 wt. %; from about 7 to about 10 wt. %, about 7 to about 9 wt. %, or any range or subrange thereof, based on the total weight of the oral care composition.

The plasticizer may modify a property of the film or for use as e.g., a further solvent, a dispersant, or as another film-forming polymer. Non-limiting examples of plasticizers include polyethylene glycol (PEG), polypropylene glycol (PPG), block polymers of polyethylene glycol and polypropylene glycol, phthalic esters, phosphoric esters, esters of adipic, azelaic, glutamic or sebacic acid, fatty acid esters citric esters, esters of acetic, propionic or butyric acid, esters of ethylbutyric or ethyl hexanoic acid, glycol esters, benzoic esters, trimellitic esters, sulfonic esters, sulfonamides, anilides, alcohols, ethers, ketones, or a combination of two or more thereof. Examples of citric acid esters include acetyl tributyl citrate and acetyl triethyl citrate.

The plasticizers may be selected from a polyethylene glycol, a polypropylene glycol, or a block polymer of polyethylene glycol and polypropylene glycol (PEG/PPG block copolymer) having a molecular weight of from about 100 to 5000 g/mol. In some embodiments, the plasticizer comprises a polypropylene glycol, a polypropylene glycol, and/or a PEG/PPG block copolymer having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600. In some embodiments, the oral care compositions may comprise one or more polyethylene glycols, e.g., polyethylene glycols in a molecular weight range from 200 to 800. For example, the compositions may comprise one or more of polyethylene glycol 200, polyethylene glycol 300, polyethylene glycol 400, polyethylene glycol 600, or polyethylene glycol 800.

Additionally or alternatively, the oral care composition may include one or more additive(s). The one or more additives may be present in the oral care composition in a total amount from about 1 to about 10 wt. %, based on the total weight of the oral care composition. In some embodiments, the oral care composition have a total amount of additives(s) from about 1 to about 10 wt. %, about 1 to about 8 wt. %, about 1 to about 6 wt. %, about 1 to about 4 wt. %, about 1 to about 3 wt. %; from about 3 to about 10 wt. %, about 3 to about 8 wt. %, about 3 to about 6 wt. %, about 3 to about 5 wt. %; from about 5 to about 10 wt. %, about 5 to about 8 wt. %, about 5 to about 7 wt. %; from about 7 to about 10 wt. %, about 7 to about 9 wt. %, or any range or subrange thereof, based on the total weight of the oral care composition.

The additives may be anti-microbial agents, anti-sensitivity agents, flavoring agents, pH adjusters, pigments and/or colorants, surfactants, or a combination of two or more thereof. The anti-microbial agents, anti-sensitivity agents, flavoring agents, pH adjusters, pigments and/or colorants, or surfactants may individually be present in the oral care composition in an amount from about 0.01 to about 7 wt. %, about 0.01 to about 5 wt. %, about 0.01 to about 4 wt. %, about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 5 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 3 wt. %, about 1 to about 2 wt. %; from about 2 to about 7 wt. %, about 2 to about 5 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %; from about 5 to about 7 wt. %, about 5 to about 6 wt. %, including any range or subrange thereof, based on the total weight of the oral care composition.

The anti-microbial agents can include, but are not limited to, chlorhexidine digluconate benzoic acid, sodium benzoate, potassium benzoate, boric acid, betanaphthol, chlorothymol, thymol, anethole, cucalyptol, carvacrol, menthol, phenol, amylphenol, hexylphenol, heptylphenol, octylphenol, hexylresorcinol, laurylpyridinium chloride, myristylpyridinium chloride, cetylpyridinium fluoride, cetylpyridinium chloride, cetylpyridinium bromide, triclosan, and a mixture of two or more thereof.

The anti-sensitivity agents can include, but are not limited, potassium salts, such as potassium nitrate, potassium bicarbonate, potassium chloride, potassium citrate, and potassium oxalate; capsaicin; eugenol; strontium salts; zinc salts; chloride salts; and a mixture of two or more thereof.

The oral care compositions may comprise one or more amino acid(s) in an effective amount. The one or more amino acids may be selected from basic amino acids, neutral amino acids, and combinations thereof. The basic amino acids may be selected from naturally occurring basic amino acids, such as arginine, lysine, and histidine, and non-naturally occurring basic amino acids having a carboxyl group and an amino group in the molecule, which are water-soluble and provide an aqueous solution with a pH of 7 or greater. Examples of basic amino acids include arginine, lysine, serine, citrulline, ornithine, creatine, histidine, diaminobutanoic acid, diaminoproprionic acid, salts thereof or combinations thereof. In some embodiments, the basic amino acids are selected from arginine, citrulline, and ornithine. In certain embodiments, the basic amino acid is arginine, e.g., L-arginine, or a salt thereof. In at least one preferred embodiment, the amino acid(s) is selected from arginine, lysine, and a combination thereof.

The one or more amino acid(s) may be present in an amount from about 0.5 to about 10 wt. %, based on the total weight of the oral care composition. In some embodiments, the one or more amino acid(s) may be present in the oral care composition in an amount from about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %; from about 6 to about 10 wt. %, about 6 to about 8 wt. %; from about 8 to about 10 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are known to one of ordinary skill in the art.

The oral care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween.

The oral care compositions may include one or more pigments, such as pigments, and/or colorants. In some embodiments, the pigments include particles ranging in size from about 0.1 μm to about 10 μm with a refractive index greater than about 1.2. Suitable pigments include, without limitation, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof.

Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti.

The surfactants can be anionic, cationic, zwitterionic, nonionic surfactants, and mixtures thereof. The one or more surfactants can include, but are not limited to: alkyl sulfates, sulfonated monoglycerides, sodium lauryl sulfate, sodium lauroyl sarcosinate, sodium coconut monoglyceride sulfonates, sodium ether lauryl, and mixtures thereof.

Any orally acceptable surfactant, most of which are anionic, nonionic, or amphoteric can be used. Examples of anionic surfactants include without limitation water-soluble salts of C₈-C₂₀ alkyl sulfates, sulfonated monoglycerides of C₈-C₂₀ fatty acids, sarcosinates, taurates, and the like. Illustrative examples of these and other classes include sodium lauryl sulfate, sodium cocoyl monoglyceride sulfonate, sodium lauroyl sarcosinate, sodium lauroyl isoethionate, sodium laureth carboxylate and sodium dodecyl benzenesulfonate. Non-limiting examples of nonionic surfactants include poloxamers, polyoxyethylene sorbitan esters, such as TWEEN™ 80, fatty alcohol ethoxylates, alkylphenol ethoxylates, tertiary amine oxides, tertiary phosphine oxides, dialkyl sulfoxides, and the like. Examples of amphoteric surfactants include, without limitation, derivatives of C₈-C₂₀ aliphatic secondary and tertiary amines having an anionic group, such as carboxylate, sulfate, sulfonate, phosphate or phosphonate. A suitable example is cocoamidopropyl betaine.

According to another aspect of the invention, provided is a method for forming a film from an oral care composition disclosed herein. Certain embodiments of the method may form a film that mineralize and/or remineralize an enamel substrate. The method may include rinsing the enamel substrate, e.g., with water; drying the enamel substrate; applying the oral care composition to the enamel substrate. The method may include applying the oral care composition with a brush applicator. In some embodiments, the oral care composition may be in the form of a spray, which is sprayed onto the enamel substrate.

### EXAMPLES

### Example 1

Five non-limiting exemplary oral care compositions (Example Compositions A-E) were prepared in accordance with aspects of the invention. Each of Example Compositions A-E were specifically formulated to form a film on an enamel substrate, e.g., a tooth. The formulations for Example Compositions A-E are shown in Table 1.

TABLE 1

| | Ex. A | Ex. B | Ex. C | Ex. D | Ex. E |
|---|---|---|---|---|---|
| US INCI Compound | (wt. %) | (wt. %) | (wt. %) | (wt. %) | (wt. %) |
| Ethanol (96 wt. % of | 53.7 | 51.2 | 48.7 | 46.2 | 56.2 |
| ethanol) | | | | | |
| Hydrogenated Rosin | 22 | 22 | 22 | 22 | 22 |
| Polyacrylate Film | 8 | 8 | 8 | 8 | 8 |
| Forming Copolymer | | | | | |
| Hydroxyapatite/ | 5 | 5 | 10 | 10 | |
| CaCO₃ | | | | | |
| Sodium Fluoride | 2.5 | 5 | 2.5 | 5 | 5 |
| Ethylcellulose | 5.5 | 5.5 | 5.5 | 5.5 | 5.5 |
| Hydroxypropyl | 2 | 2 | 2 | 2 | 2 |
| Cellulose | | | | | |
| Saccharin | 0.7 | 0.7 | 0.7 | 0.7 | 0.7 |
| Flavor | 0.6 | 0.6 | 0.6 | 0.6 | 0.6 |

A comparative composition (Comparative Composition 1) was prepared according to the formula shown in Table 2.

TABLE 2

| | Comparative Composition 1 |
|---|---|
| US INCI Compound | (wt. %) |
| Ethanol (96 wt. % of | 56.2 |
| ethanol) | |
| Hydrogenated Rosin | 22 |
| Polyacrylate Film | 8 |
| Forming Copolymer | |
| Hydroxyapatite | 5 |
| Sodium Fluoride | |
| Ethylcellulose | 5.5 |
| Hydroxypropyl | 2 |
| Cellulose | |
| Saccharin | 0.7 |
| Flavor | 0.6 |

### Example 2

An in vitro enamel protection model was utilized to evaluate the effect of Example Compositions B and E in comparison to Comparative Composition 1. Enamel blocks were prepared to have an enamel layer of about 1 mm thickness and a dentin layer of about 1 mm thickness. Before the application of Example Composition B, Example Composition E, or Comparative Composition 1, the enamel blocks were hydrated overnight by submerging the enamel blocks in 8 ml of remineralization solution. The enamel blocks were removed from the remineralization solution the next afternoon, rinsed with water for 2 minutes, and air dried for 5 minutes.

A sample of each of Example Composition B, Example Composition E, and Comparative Composition 1 was then applied to respective bovine enamel blocks using a micro brush and subsequently allowed to air dry for 5 minutes. Each enamel block was placed again into a remineralization solution and kept in an incubator at a temperature of 37° C. overnight. The enamel blocks were removed from the remineralization solution the next morning, cleaned, and evaluated.

The enamel blocks were then submerged into an acid solution comprising 1 wt. % of citric acid and having a pH of 3.5 for 2 minutes. The enamel blocks were then rinsed with water. The enamel blocks were subsequently submerged into a remineralization solution for 1 hour and then rinsed. The enamel blocks were then resubmerged into an acid solution, rinsed, submerged in the remineralization solution and then rinsed. The process of submerging in an acid solution, rinsing, submerging into a remineralization solution and then rinsing was completed a total of four times before the enamel blocks were evaluated.

The microhardness of the bovine enamel blocks was evaluated using a Knoop microhardness tester (50 gram load, 15 seconds, 5 measurements per block). As enamel is exposed to acid, the outer layer begins to dissolve resulting in mineral loss and structural weakening, and/or demineralization, which results in a reduction of microhardness. The final microhardness value was measured with indentation in close proximity with previous indent using a 50 gram load for 15 seconds (5 measurements per block). The % demineralization was calculated according to the formula below to represent the mineral loss after the acid treatment. A Tukey Test was applied for statistical analysis.

$$\% Hardness Repair = \frac{Sound Hardness - Final Hardness}{Sound Hardness}$$

Profilometry measurements were taken for each enamel block. Specifically, half of each enamel block was covered with tape as a reference side before the acid treatment of the enamel blocks. When enamel is exposed to acid, the enamel surface is etched away. It is possible to protect a portion of the enamel while etching the other side to determine the loss of enamel during acid exposure. Utilizing the step height measurements, tissue loss caused by acid treatment was quantified.

After the preparation and acid treatment of the enamel blocks, the tape was removed and the enamel block surface was cleaned by using a cotton swab with acetone and ethanol. The surface roughness of the acid treated side and the step height difference between the reference side (covered by tape) and the acid treated side of each enamel block was measured by a Keyence microscope at 50× magnification. The surface and cross-sectional morphologies and cross section of the treated samples were observed using a scanning electron microscopy (SEM), namely a Phenom ProX Desktop SEM.

X-ray Photoelectron Spectroscopy (XPS or ESCA) was used to determine the surface chemical composition of the enamel block samples treated with the oral care compositions. ESCA analyses were conducted using a PHI VersaProbe II Scanning XPS Microprobe (Ulvac PHI, Chanhassen, MN). Samples were analyzed using a 100 W Al K alpha monochromatic x-ray, 1200×100 micron spot size, 45 degree tilt, and a 29 eV analyzer pass energy. Sample charge neutralization was accomplished using a dual beam system of low energy electrons and Ar+ ions. Replicate samples of the oral care composition treated enamel and the control (water was applied instead of an oral care composition), were studied at three separate areas per sample.

As mentioned above, the effect of oral care composition on enamel demineralization post acid treatment can be measured by the loss of enamel's hardness. This allows for the evaluation of each oral care composition's performance or efficacy at protecting enamel from acid erosion. A better oral care composition will show a low demineralization percentage, as the enamel is protected from the acid by the oral care composition. Conversely, an oral care composition with a higher demineralization value will not protect the enamel from acid attack as effectively. Table 3 shows the results of the demineralization of the enamel blocks. The treatments were given a different grouping letter when the demineralization percent was significantly different.

TABLE 3

| | % Demineralization ± | |
|---|---|---|
| Treatment | StDev | Grouping* |
| Water (control) | 47.02 ± 4.11 | A |
| Comparative Composition 1 | 33.10 ± 4.58 | C |
| Example Composition E | 27.25 ± 2.45 | B |
| Example Composition B | 21.19 ± 2.72 | D |

The results of the Profilometry measurements were assessed by way of evaluating step height changes for the enamel blocks, which are provided in Table 4. A higher surface roughness is generally indicative of more acid damage as the outer layer of enamel has begun to wear away. As noted above, the treatments were given a different grouping letter when the demineralization percent was significantly different.

TABLE 4

| | Step Height ± | |
|---|---|---|
| Treatment | StDev | Grouping* |
| Water (control) | 1.07 ± 0.36 | A |
| Comparative Composition 1 | 0.58 ± 0.12 | B |
| Example Composition E | 0.29 ± 0.13 | BC |
| Example Composition B | 0.21 ± 0.08 | C |

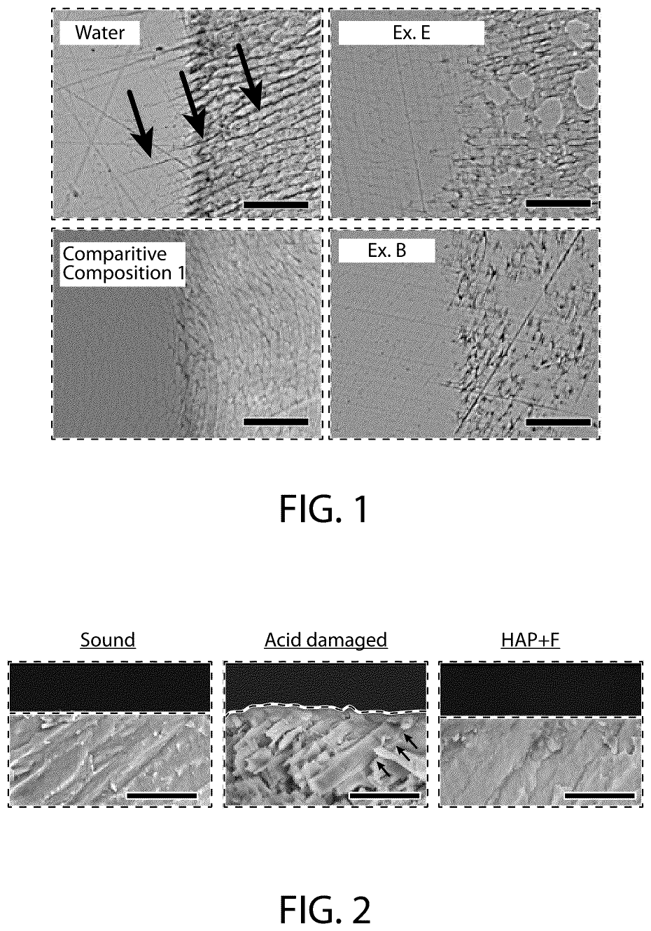

Images of the enamel blocks from the scanning electron microscopy are shown in FIG. 1. As seen in FIG. 1, clear prism gaps were observed on the surface of enamel blocks that received water (the control) instead of Example Composition B, Example Composition E, or Comparative Composition 1. FIG. 2 is a cross-sectional view of a sound enamel block, acid damaged enamel block, and enamel block receiving Example Composition B.

Table 5 provides the results of the XP analysis.

TABLE 5

| | Atomic Percentages | | | | | | | Ratios | | |
|---|---|---|---|---|---|---|---|---|---|---|
| | C | O | N | Na | Ca | P | F | P/Ca | F/Ca | F Peak |
| Water | 17.6 | 53.4 | 0.74 | 0.26 | 15.8 | 11.7 | 0.53 | 0.74 | 0.034 | 1341 |
| (Control) | | | | | | | | | | |
| Comparative | 29.2 | 46.3 | 0.95 | 0.21 | 13.1 | 9.6 | 0.46 | 0.73 | 0.035 | 1341 |
| Composition | | | | | | | | | | |
| Example | 34.5 | 40.1 | 1.19 | 0.21 | 10.9 | 8.1 | 1.02 | 0.75 | 0.094 | 1341 |
| Composition E | | | | | | | | | | |
| Example | 30 | 45.6 | 0.79 | 0.29 | 13 | 9.6 | 1.28 | 0.74 | 0.099 | 1341 |
| Composition B | | | | | | | | | | |

As seen in Table 5, the fluoride concentrations and F/Ca ratios for the enamel blocks receiving Example Composition B and Example Composition E were similar and greater than those for Comparative Composition 1 and water controls, indicating fluoride delivery to the enamel surfaces. Fluoride peak separation data suggests that the fluoride detected was primarily fluorapatite.

### Example 3

Example Composition E was evaluated in comparison to a commercial benchmark (Comparative Composition 2) to assess the fluoride uptake on an enamel substrate. Comparative Composition 2 had the following ingredients: sodium fluoride, ethanol, white beeswax, shellac, colophony, mastic, saccharin, and flavor.

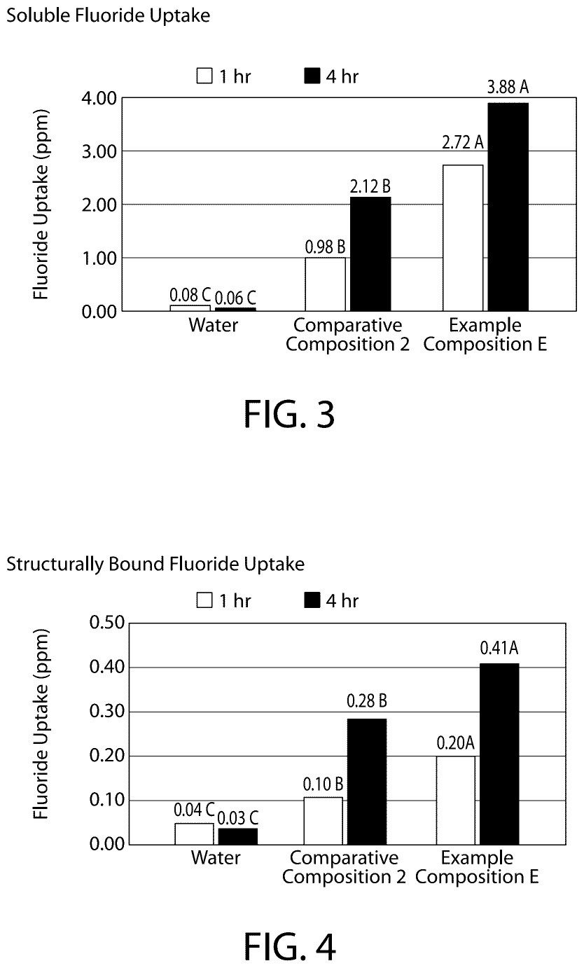

A sample of Example Composition E and Comparative Composition 2 were applied to bovine enamel blocks and incubated in 2 ml of artificial saliva for 4 hours at 37° C. The enamel blocks were then rinsed with water and the film formed from Example Composition E and Comparative Composition 2 was removed with acetone/ethanol. The blocks were then soaked in 1 ml of 1 M potassium hydroxide for 24 hours. After 24 hours, the enamel blocks were removed, rinsed with water, and soaked in 1 ml or 1 M perchloric acid for 15 min. Collected samples were measured for fluoride analysis using an ion selective probe. The results of the fluoride analysis are shown in FIGS. 3 and 4.

As seen in FIGS. 3 and 4, Example Composition E delivered statistically more fluoride uptake as compared to Comparative Composition 2, at both 1 and 4 hour time points. Soluble fluoride is representative of the fluoride found on the surface, calcium fluoride (CaF₂) layer. Structurally bound fluoride is representative of the amount of fluoride that is extracted from beneath the enamel surface, namely, the fluorapatite (FAP) layer.

### Example 4

An in vitro enamel protection model was utilized to evaluate the effect Example Composition E, Comparative Composition 2, and another commercial benchmark composition (Comparative Composition 3). Comparative Composition 3 had the following list of ingredients: sodium fluoride, ethanol, pentaerythritol glycerol ester of colophony resin, n-hexane, thickener, flavor enhancer, flavor, and modified tricalcium phosphate.

The in vitro enamel protection model was conducted according to similar procedures as those described in Example 1. For instance, Example Composition E, Comparative Composition 2, and Comparative Composition 3 were applied to enamel blocks to determine the demineralization of the enamel blocks based on the microhardness of such blocks. Table 6 provides a summary of the results of the demineralization of the blocks receiving Example Composition E, Comparative Composition 2, and Comparative Composition 3. The treatments were given a different grouping letter when the demineralization percent was significantly different. It is important to note that the absolute value of demineralization cannot be compared across different experiments or Examples as the enamel block batches and reaction to acid treatments often differ by experiment.

TABLE 6

| | % Demineralization ± | |
|---|---|---|
| Treatment | StDev | Grouping* |
| Example | 31.43 ± 4.73 | C |
| Composition E | | |
| Comparative | 41.19 ± 3.15 | B |
| Composition 2 | | |
| Comparative | 51.22 ± 7.83 | A |
| Composition 3 | | |

A summary of the results of the ICP analysis, which determined the calcium content is provided in Table 7. The treatments were given a different grouping letter when the Ca concentration was significantly different.

TABLE 7

| | Ca concentration (ppm) ± | |
|---|---|---|
| Treatment | StDev | Grouping* |
| Example | 3.61 ± 0.98 | C |
| Composition E | | |
| Comparative | 6.34 ± 0.85 | A |
| Composition 2 | | |
| Comparative | 6.18 ± 2.40 | A |
| Composition 3 | | |

As seen in Table 7, less calcium from the film formed of Example Composition E was dissolved by the acid treatment, which indicates that Example Composition E provides better enamel protection than Comparative Composition 2 or Comparative Composition 3.

A summary of the tissue loss determined from the step height measurements are shown in table 8. The treatments were given a different grouping letter when the step height was significantly different.

TABLE 8

| Treatment | Step height (μm) ± StDev | Grouping* |
|---|---|---|
| Water | 6.26 ± 0.733 | A |
| Example | 0.38 ± 0.223 | C |
| Composition E | | |
| Comparative | 1.59 ± 1.141 | C |
| Composition 2 | | |
| Comparative | 3.78 ± 1.218 | B |
| Composition 3 | | |

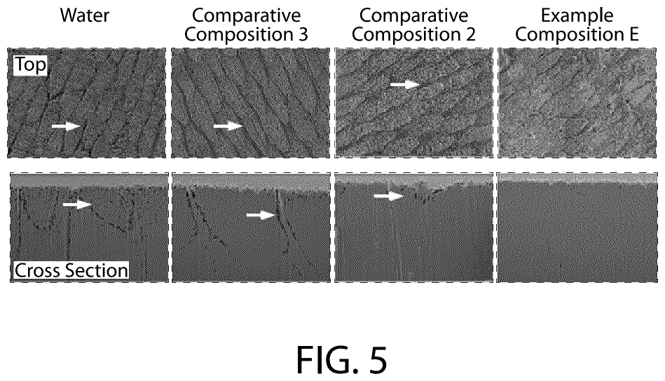

Enamel blocks were imaged by scanning electron microscopy as well as focused ion beam cross-sectioning to study the surface and subsurface changes due to the treatments. FIG. 5 is a top view and cross-sectional images of the enamel blocks receiving Example Composition E, Comparative Composition 2, or Comparative Composition 3.

### Example 5

An in vitro enamel protection model was utilized to evaluate the effect Example Composition E and another commercial benchmark composition (Comparative Composition 4). Comparative Composition 4 had the following list of ingredients: sodium fluoride, ethyl alcohol, hydrogenated rosin, hexadecane phosphate, flavor, citric acid, polysorbate 80, sucralose, and xylitol.

The in vitro enamel protection model was conducted according to similar procedures as those described in Example 1. For instance, Example Composition E and Comparative Composition 4 were applied to enamel blocks to determine the demineralization of the enamel blocks based on the microhardness of such blocks.

Table 9 provides a summary of the results of the demineralization of the blocks receiving Example Composition E and Comparative Composition 4. The treatments were given a different grouping letter when the demineralization percent was significantly different. It is important to note that the absolute value of demineralization cannot be compared across different experiments or Examples as the enamel block batches and reaction to acid treatments often differ by experiment.

TABLE 9

| | % Demineralization ± | |
|---|---|---|
| Treatment | StDev | Grouping* |
| Water (Control) | 44.00 ± 5.09 | A |
| Example | 19.74 ± 4.96 | C |
| Composition E | | |
| Comparative | 29.26 ± 5.16 | B |
| Composition 4 | | |

### Example 4

Five compositions (Compositions 5-9) were prepared having formulations shown in Table 10.

TABLE 10

| | Ex. E | Comp. 5 | Comp. 6 | Comp. 7 | Comp. 8 | Comp. 9 |
|---|---|---|---|---|---|---|
| US INCI Compound | (wt. %) | (wt. %) | (wt. %) | (wt. %) | (wt. %) | (wt. %) |
| Ethanol (96 wt. % of | 56 | 64 | 78 | 60 | 58 | 58 |
| ethanol) | | | | | | |
| Hydrogenated Rosin | 22 | 22 | | 22 | 22 | 22 |
| Polyacrylate Film | 8 | | 8 | 8 | 8 | 8 |
| Forming Copolymer | | | | | | |
| Sodium Fluoride | 5 | 5 | 5 | 5 | 5 | 5 |
| Ethylcellulose having | 4 | 4 | 4 | 4 | | 4 |
| 50 cps | | | | | | |
| Ethylcellulose having | 1.5 | 1.5 | 1.5 | | 1.5 | 1.5 |
| 10 cps | | | | | | |
| Hydroxypropyl | 2 | 2 | 2 | 2 | 2 | |
| Cellulose | | | | | | |
| Saccharin | 0.7 | 0.7 | 0.7 | 0.7 | 0.7 | 0.7 |
| Flavor | 0.6 | 0.6 | 0.6 | 0.6 | 0.6 | 0.6 |

### Example 5

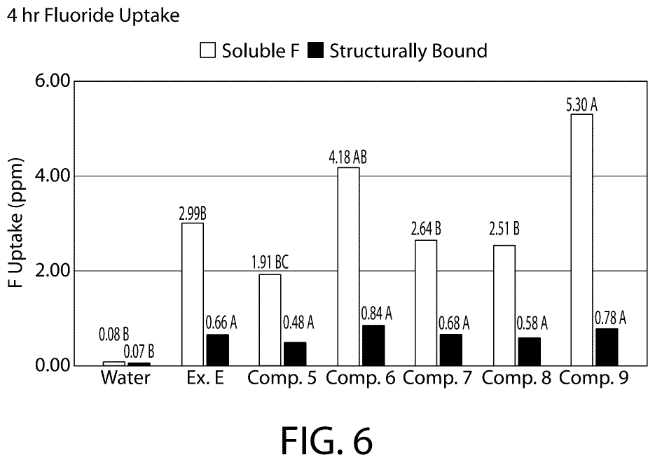

Samples of Example Composition E and Compositions 5-9 were each evaluated for fluoride uptake using a procedure similar to that described in Example 3. The results of the fluoride uptake are shown in FIG. 6.

### Example 6

Two exemplary oral care compositions (Example Compositions F and G) were prepared in accordance with aspects of the invention. The formulation for Exemplary Compositions F and G are shown in Table 11.

TABLE 11

| | Ex. F | Comp. G |
|---|---|---|
| US INCI Compound | (wt. %) | (wt. %) |
| Ethanol (96 wt. % of ethanol) | QS to 100 | QS to 100 |
| Hydrogenated Rosin | 22 | 22 |
| Polyacrylate Film Forming | 8 | 8 |
| Copolymer | | |
| Functionalized Hydroxyapatite | 5 | 5 |
| (Hydroxyapatite/CaCO₃) | | |
| Sodium Fluoride | 5 | 5 |
| Ethylcellulose having 50 cps | 1.5 | 1.5 |
| Ethylcellulose having 10 cps | 4 | 4 |
| Hydroxypropyl Cellulose | 2 | 2 |
| Saccharin | 0.7 | 0.7 |
| Flavor | 0.7 | 0.7 |
| Polyvinylpyrrolidone | | 2 |

### Example 7

A solid-state NMR study was conducted to evaluate the interactions and HAP and sodium fluoride in oral care compositions according to aspects of the disclosure. Specifically, Example Composition F was evaluated in comparison to a control composition (Control Composition), which differed from Example Composition F in that the Control Composition did not include the functionalized hydroxyapatite. From the NMR results, it was determined that Example Composition F exhibited a sodium fluoride peak matching the Control Composition at −223 ppm. Additionally, the formation of CaF2 or fluorapatite was not observed in Example Composition F. Accordingly, although it may have been conventionally believed that HAP would interact and/or react with the sodium fluoride to reduce the amount of fluoride that can be delivered to teeth, Exemplary Composition F unexpectedly exhibited a similar amount of deliverable fluoride as the Control Composition, which did not include hydroxyapatite.

What is claimed is:

1. An oral care composition comprising:
from about 1 to about 10 wt. % of a fluoride ion source;
from about 2 to about 20 wt. % of hydroxyapatite;
from about 3 to about 30 wt. % of a film-forming agent; and
from about 7 to about 35 wt. % of an adhesive,
wherein the oral care composition is adapted to form a film on teeth, and all weight percentages are based on the total weight of the oral care composition.

2. The oral care composition according to claim 1, wherein the oral care composition delivers from about 5,000 to about 48,000 ppm of fluoride ions.

3. The oral care composition according to claim 1, wherein the film-forming agent is selected from an acrylamide polymer, an acrylate polymer, a cellulose, a cellulose derivative, a gum, a pyrrolidone polymer, and a combination of two or more thereof.

4. The oral care composition according to claim 1, wherein the hydroxyapatite comprises nano-hydroxyapatite.

5. The oral care composition according to claim 1, wherein the hydroxyapatite comprises micro-hydroxyapatite.

6. The oral care composition according to claim 1, wherein the hydroxyapatite is functionalized.

7. The oral care composition according to claim 1, wherein the film-forming agent comprises a hydroxypropylmethyl cellulose, hydroxyethyl cellulose, hydroxypropyl cellulose, polyvinyl pyrrolidone, carboxymethyl cellulose, sodium carboxy methyl cellulose, methyl cellulose, polyvinyl alcohol, sodium alginate, polyethylene glycol, polyethylene oxide, chitosan, xanthan gum, tragacantha, guar gum, acacia gum, arabic gum, carrageenan, pululan, polyacrylic acid, methylmethacrylate copolymer, carboxyvinyl copolymers, octylacrylamide copolymer, or a combination of two or more thereof.

8. The oral care composition according to claim 1, wherein the adhesive is selected from a rosin, a polyvinyl acetaldehyde, a polyvinyl alcohol, a polyvinyl acetate, a poly(ethylene oxide), a polyacrylate, a polyvinylpyrrolidone, a polyvinylpyrrolidone/vinyl acetate copolymer, a polyoxyethylene/polyoxypropylene block copolymer, a silicone resin, an organo phosphoric acid having alkyl group, and a combination of two or more thereof.

9. The oral care composition according to claim 8, wherein the rosin is selected from a wood rosin, a gum rosin, a tall oil rosin, and a combination of two or more thereof.

10. The oral care composition according to claim 9, wherein the rosin is at least partially hydrogenated or fully hydrogenated.

11. The oral care composition according to claim 8, wherein the adhesive comprises a polyvinylpyrrolidone, a polyvinylpyrrolidone/vinyl acetate copolymer, or a combination thereof.

12. The oral care composition according to claim 1 further comprising:
from about 20 to about 70 wt. % of an orally acceptable vehicle.

13. The oral care composition according to claim 12, wherein the orally acceptable vehicle comprises a volatile component selected from one or more of ethyl acetate i-pentyl propionate, acetone, ethylacetate i-amylpropionate, hydrogen peroxide, methanol, ethanol, propanol, isopropanol, butanol, and a combination of two or more thereof.

14. The oral care composition according to claim 1 further comprising:
one or more plasticizer selected from citric acid esters.

15. The oral care composition according to claim 14, wherein the citric acid ester is selected from acetyl tributyl citrate, acetyl triethyl citrate, and a combination of two or more thereof.

16. The oral care composition according to claim 1, wherein the oral care composition comprises a weight ratio of the film-forming agent to the adhesive from about 1:1 to about 1:5.

17. The oral care composition according to claim 1, wherein the oral care composition is free of water.

18. The oral care composition according to claim 1, wherein the oral care composition is adapted to form a film on an enamel substrate within 2 minutes from application to the enamel substrate.

19. An oral care composition comprising:
from about 1 to about 10 wt. % of a fluoride ion source;
from about 3 to about 30 wt. % of a film-forming agent; and
from about 7 to about 35 wt. % of an adhesive,
wherein the oral care composition is adapted to form a film on teeth, and all weight percentages are based on the total weight of the oral care composition.

20. An oral care composition comprising:
from about 1 to about 10 wt. % of a fluoride ion source;
optionally, from about 2 to about 20 wt. % of hydroxyapatite;
from about 3 to about 30 wt. % of a film-forming agent;
from about 7 to about 35 wt. % of an adhesive; and
a polyvinylpyrrolidone,
wherein the oral care composition is adapted to form a film on teeth, and all weight percentages are based on the total weight of the oral care composition.
